(19) United States
(12) Patent Application Publication
(10) Pub. No.: US 2011/0091451 A1
(43) Pub. Date: Apr. 21, 2011

(54) Methods for preventing and treating cancer metastasis and bone loss associated with cancer metastasis

(76) Inventors: William M. Kavanaugh, Orinda, CA (US); Gregory M. Harrowe, Eugene, OR (US); Kirston Koths, El Cerrito, CA (US); Cheng Liu, Richmond, CA (US); Deborah L. Zimmerman, Oakland, CA (US)

(21) Appl. No.: 10/914,598

(22) Filed: Aug. 9, 2004

Related U.S. Application Data
(63) Continuation of application No. 10/713,895, filed on Nov. 15, 2002, now abandoned.

(51) Int. Cl. A61K 39/395; A61P 35/00; A61P 19/08

(52) U.S. Cl. 424/133.1

(57) ABSTRACT

M-CSF antagonists are used to prepare compositions, including pharmaceutical compositions, for preventing or treating cancer metastasis and/or bone loss associated with cancer metastasis in a mammal.

### BACKGROUND OF THE INVENTION

1. Field of the Invention

This invention relates to methods for preventing and treating cancer metastasis and bone loss associated with cancer metastasis by administering M-CSF antagonist to a mammal.

2. Description of the Related Art

Cancer metastasis is the primary cause of post-operation or post-therapy recurrence in cancer patients. Despite intensive efforts to develop treatments, cancer metastasis remains substantially refractory to therapy. Bone is one of the most common sites of metastasis of various types of human cancers (e.g., breast, lung, prostate and thyroid cancers). The occurrence of osteolytic bone metastases causes serious morbidity due to intractable pain, high susceptibility to fracture, nerve compression and hypercalcemia. Despite the importance of these clinical problems, there are few available treatments for bone loss associated with cancer metastasis.

Osteoclasts mediate bone readsorption. Osteoclasts are multinucleated cells differentiating from haemopoietic cells. It is generally accepted that osteoclasts are formed by the fusion of mononuclear precursors derived from haemopoietic stem cells in the bone marrow, rather than incomplete cell divisions (Chambers, Bone and Mineral Research, 6: 1-25, 1989; Göthling et al., Clin Orthop Relat R. 120: 201-228, 1976; Kahn et al., Nature 258: 325-327, 1975, Suda et al., Endocr Rev 13: 66-80, 1992; Walker, Science 180: 875, 1973; Walker, Science 190: 785-787, 1975; Walker, Science 190: 784-785, 1975). They share a common stem cell with monocyte-macrophage lineage cells (Ash et al., Nature 283: 669-670, 1980, Kerby et al., J. Bone Miner Res 7: 353-62, 1992). The differentiation of osteoclast precursors into mature multinucleated The differentiation of osteoclast precursors into mature multinucleated osteoclasts requires different factors including hormonal and local stimuli (Athanasou et al., Bone Miner 3: 317-333, 1988; Feldman et al., Endocrinology 107: 1137-1143, 1980; Walker, Science 190: 784-785, 1975; Zheng et al., Histochem J 23: 180-188, 1991) and living bone and bone cells have been shown to play a critical role in osteoclast development (Hagenaars et al., Bone Miner 6: 179-189, 1989). Osteoblastic or bone marrow stromal cells are also required for osteoclast differentiation. One of the factors produced by these cells that support osteoclast formation is macrophage colony-stimulating factor, M-CSF (Wiktor-Jedrzejczak et al., Proc Natl Acad Sci USA 87: 4828-4832, 1990; Yoshida et al., Nature 345: 442-444, 1990). Receptor activator for NF-K B ligand (RANKL, also known as TRANCE, ODF and OPGL) is another signal (Suda et al., Endocr Rev 13: 66-80, 1992) through which osteoblastic/stromal cells stimulate osteoclast formation and resorption via a receptor RANK (TRANCER) located on osteoclasts and osteoclast precursors (Lacey et al., Cell 93: 165-176, 1998; Tsuda et al., Biochem Biophys Res Co 234: 137-142, 1997; Wong et al., J Exp Med 186: 2075-2080, 1997; Wong et al., J Biol. Chem 272: 25190-25194, 1997; Yasuda et al., Endocrinology 139: 1329-1337, 1998; Yasuda et al., Proc Natl Acad Sci US 95: 3597-3602, 1998). Osteoblasts also secrete protein that strongly inhibits osteoclast formation called osteoprotegerin (OPG, also known as OCIF), which acts as a decoy receptor for the RANKL thus inhibiting the positive signal between osteoclasts and osteoblasts via RANK and RANKL.

Osteoclasts are responsible for dissolving both the mineral and organic bone matrix (Blair et al., J Cell Biol 102: 1164-1172, 1986). Osteoclasts represent terminally differentiated cells expressing a unique polarized morphology with specialized membrane areas and several membrane and cytoplasmic markers, such as tartrate resistant acid phosphatase (TRAP) (Anderson et al. 1979), carbonic anhydrase II (Väänänen et al., Histochemistry 78: 481-485, 1983), calcitonin receptor (Warshafsky et al., Bone 6: 179-185, 1985) and vitronectin receptor (Davies et al., J Cell Biol 109: 1817-1826, 1989). Multinucleated osteoclasts usually contain less than 10 nuclei, but they may contain up to 100 nuclei being between 10 and 100 μm in diameter (Göthling et al., Clin Orthop Relat R 120: 201-228, 1976). This makes them relatively easy to identify by light microscopy. They are highly vacuolated when in active state, and also contain many mitochondria, indicating a high metabolic rate (Mundy, in Primer on the metabolic bone diseases and disorders of mineral metabolism, pages 18-22, 1990). Since osteoclasts play a major role in osteolytic bone metastases, there is a need in the art for new agents and methods for preventing osteoclast stimulation.

Thus, there remains a need to identify new agents and methods for preventing or treating cancer metastasis, including osteolytic bone metastases. The compositions and methods of the present invention fulfill these and other related needs.

### SUMMARY OF THE INVENTION

In one aspect, the present invention provides methods for treating bone loss associated with cancer metastasis, which methods comprise the administration of a M-CSF antagonist or pharmaceutical composition thereof. In a related aspect, the present invention provides methods for preventing the development of a metastastic cancer to bone, which methods also comprise the administration of a M-CSF antagonist or pharmaceutical composition thereof. More specifically, an amount of a M-CSF antagonist or pharmaceutical composition thereof is administered to a patient afflicted with, or predisposed to, a metastatic cancer to bone and thereby inhibits the interaction between M-CSF and its receptor (M-CSF R).

In another aspect, the present invention provides methods for preventing or treating cancer metastasis. These methods comprise administering to a mammal afflicted with a cancer a therapeutically effective amount of a M-CSF antagonist thereby preventing metastasis of the cancer or reducing the severity of metastasis of the cancer (e.g., reducing the number or sizes of metastases).

By the present invention, M-CSF antagonists may be monoclonal or polyclonal antibodies, including humanized or human antibodies. Alternatively, M-CSF antagonists include suitable proteins or peptides or other small molecules that bind to M-CSF thereby inhibiting the interaction of M-CSF and M-CSF R. The M-CSF antagonist compositions can be formulated in amounts sufficient to reverse or diminish the severity of bone loss associated with cancer metastasis or to prevent or treat cancer metastasis in cancer patients. The compositions of the present invention may further comprise a pharmaceutically acceptable carrier or stabilizer suitable for in vivo administration. In some embodiments, these compositions may be further combined with additional agents efficacious against cancer metastasis or bone loss associated with cancer metastasis.

The present invention thus provides the art with compositions and methods that are effective in treating cancer metastasis and bone loss associated with cancer metastasis.

The invention provides a method for treating a mammal afflicted with a metastatic cancer to bone comprising administering to the mammal a therapeutically effective amount of M-CSF antagonist thereby reducing the severity of bone loss associated with the metastatic cancer.

The invention further provides a method for preventing the development of a metastatic cancer to bone comprising administering to a mammal predisposed to a metastatic cancer to bone a therapeutically effective amount of M-CSF antagonist thereby preventing the development of a metastatic cancer to bone.

In specific embodiments, the antagonist blocks the interaction between M-CSF and M-CSF R, and in a more specific embodiment, the antagonist is an antibody that binds to M-CSF.

According to the methods of the invention, the antibody may be a monoclonal antibody, and in one embodiment the antibody is a humanized monoclonal antibody.

In another embodiment, the antibody is a human monoclonal antibody.

In a specific embodiment, the antibody is 5H4.

In another specific embodiment, the antagonist is a fragment of antibody 5H4.

The invention also provides a method for treating a mammal afflicted with a metastatic cancer to bone comprising administering to the mammal a therapeutically effective amount of M-CSF antagonist thereby reducing the severity of bone loss associated with the metastatic cancer, wherein the mammal is human.

In certain embodiments, the M-CSF antagonist is administered intraperitoneally or intradermally.

The invention further provides a method for preventing or treating cancer metastasis, comprising administering to a mammal afflicted with a cancer a therapeutically effective amount of a M-CSF antagonist thereby preventing metastasis of the cancer or reducing the severity of metastasis of the cancer.

### BRIEF DESCRIPTION OF THE SEVERAL VIEWS OF THE DRAWINGS

FIG. 1 is a comparison of osteoclast inducing activity between purified M-CSF and conditioned medium (CM) from MDA-231 cells and MCF7 cells.

FIG. 2 is a comparison of the neutralizing activity of a monoclonal antibody 5H4 against purified M-CSF relative to CM from MDA231 cells.

FIG. 3 shows neutralizing activity of 5H4 and other antibodies against human M-CSF.

### DETAILED DESCRIPTION OF THE INVENTION

The ability to metastasize is a defining characteristic of a cancer. Metastasis refers to the spread of cancer cells to other parts of the body or to the condition produced by this spread. Metastasis is a complex multistep process that includes changes in the genetic material of a cell, uncontrolled proliferation of the altered cell to form a primary tumor, development of a new blood supply for the primary tumor, invasion of the circulatory system by cells from the primary tumor, dispersal of small clumps of primary tumor cells to other parts of the body, and the growth of secondary tumors in those sites.

Bone is one of the most common sites of metastasis in human breast, lung, prostate and thyroid cancers, as well as other cancers, and in autopsies as many as 60% of cancer patients are found to have bone metastasis. Osteolytic bone metastasis shows a unique step of osteoclastic bone resorption that is not seen in metastasis to other organs. Bone loss associated with cancer metastasis is mediated by osteoclasts (multinucleated giant cells with the capacity to resorb mineralized tissues), which seem to be activated by tumor products.

Colony stimulating factor (CSF-1), also known as macrophage colony stimulating factor (M-CSF), has been found crucial for osteoclast formation. In addition, M-CSF has been shown to modulate the osteoclastic functions of mature osteoclasts, their migration and their survival in cooperation with other soluble factors and cell to cell interactions provided by osteoblasts and fibroblasts. Fixe and Praloran, Cytokine 10: 3-7, 1998; Martin et al., Critical Rev. in Eukaryotic Gene Expression 8: 107-23, 1998.

The full-length human M-CSF mRNA encodes a precursor protein of 554 amino acids (Accession No. FQHUMP). Through alternative mRNA splicing and differential post-translational proteolytic processing, M-CSF can either be secreted into the circulation as a glycoprotein or chondroitin sulfate containing proteoglycan or be expressed as a membrane spanning glycoprotein on the surface of M-CSF producing cells. The three-dimensional structure of the bacterially expressed amino terminal 150 amino acids of human M-CSF, the minimal sequence required for full in vitro biological activity, indicates that this protein is a disulfide linked dimer with each monomer consisting of four alpha helical bundles and an anti-parallel beta sheet. Pandit et al., Science 258: 1358-62, 1992. Three distinct M-CSF species are produced through alternative mRNA splicing. The three polypeptide precursors are M-CFSα of 256 aa, M-CSFβ of 554 aa, and M-CSFγ of 438 aa. M-CSFβ is a secreted protein that does not occur in a membrane-bound form. M-CSFα is expressed as an integral membrane protein that is slowly released by proteolytic cleavage. The membrane-bound form of M-CSF can interact with receptors on nearby cells and therefore mediates specific cell-to-cell contacts.

Various forms of M-CSF function by binding to its receptor M-CSFR on target cells. M-CSFR is a membrane spanning molecule with five extracellular immunoglobulin-like domains, a transmembrane domain and an intracellular interrupted Src related tyrosine kinase domain. M-CSFR is encoded by c-fms proto-oncogene. Binding of M-CSF to the extracellular domain of M-CSFR leads to dimerization of the receptor, which activates the cytoplasmic kinase domain, leading to autophosphorylation and phosphorylation of other cellular proteins. Hamilton, Trends Immunol. Today 18: 3137, 1997. Phosphorylated cellular proteins induce a cascade of biochemical events leading to cellular responses: mitosis, secretion of cytokines, membrane ruffling, and regulation of transcription of its own receptor. Fixe and Praloran, Cytokine 10: 32-37, 1998.

M-CSF is expressed in stromal cells, osteoblasts, and other cells. It is also expressed in breast, uterine, and ovarian tumor cells. The extent of expression in these tumors correlates with high grade and poor prognosis. Kacinski Ann. Med. 27: 79-85, 1995; Smith et al., Clin. Cancer Res. 1: 313-25, 1995. In breast carcinomas, M-CSF expression is prevalent in invasive tumor cells as opposed to the intraductal (preinvasive) cancer. Scholl et al., J. Natl. Cancer Inst. 86: 120-6, 1994. In addition, M-CSF is shown to promote progression of mammary tumors to malignancy. Lin et al., J. Exp. Med. 93: 727-39, 2001. For breast and ovarian cancer, the production of M-CSF seems to be responsible for the recruitment of macrophages to the tumor.

Even with the above understanding of the relationship between M-CSF and cancer metastasis as well as the function of M-CSF in osteoclast formation, to the knowledge of the inventors of the present invention, there exists no report of using a M-CSF antagonist in preventing or treating cancer metastasis or bone loss associated with cancer metastasis. It has been discovered, as part of the present invention, that M-CSF antagonists neutralize osteoclast induction by metastatic cancer cells. Thus, the present invention provides compositions and methods for treating or preventing cancer metastasis and bone loss associated with cancer metastasis.

As used herein, the term “antagonist” generally refers to the property of a molecule, compound or other agent to, for example, interfere with the binding of one molecule with another molecule or the stimulation of one cell by another cell either through steric hindrance, conformational alterations or other biochemical mechanism. In one regard, the term antagonist relates to the property of an agent to prevent the binding of a receptor to its ligand, e.g., the binding of M-CSF with M-CSF R, thereby inhibiting the signal transduction pathway triggered by M-CSF. The term antagonist is not limited by any specific action mechanism, but, rather, refers generally to the functional property presently defined. Antagonists of the present invention include, but are not limited to, antibodies or peptides as well as other molecules that bind to M-CSF.

Effective therapeutics depend on identifying efficacious agents devoid of significant toxicity. Compounds potentially useful in preventing or treating bone loss associated with cancer metastasis may be screened using various assays. For instance, a candidate antagonist may first be characterized in a cultured cell system to determine its ability to neutralize M-CSF in inducing osteoclastogenesis. Such a system may include the co-culture of mouse calvarial osteoblasts and spleen cells (Suda et al., Modulation of osteoclast differentiation. Endocr. Rev. 13: 66-80, 1992; Martin and Udagawa, Trends Endocrinol. Metab. 9: 6-12, 1998), the co-culture of mouse stromal cell lines (e.g., MC3T3-G2/PA6 and ST2) and mouse spleen cells (Udagawa et al., Endocrinology 125: 1805-13, 1989), and the co-culture of ST2 cells and bone marrow cells, peripheral blood mononuclear cells or alveolar macrophages (Udagawa et al., Proc. Natl. Acad. Sci. USA 87: 7260-4, 1990; Sasaki et al., Cancer Res. 58: 462-7, 1998; Mancino et al., J. Surg. Res. 100: 18-24, 2001). In the absence of any M-CSF antagonist, multinucleated cells formed in such co-cultures satisfy the major criteria of osteoclasts such as tartrate resistant acid phosphatase (TRAP, a marker enzyme of osteoclasts) activity, calcitonin receptors, p60^{C-STC}, vitronectin receptors, and the ability to form resorption pits on bone and dentine slices. The presence of an effective M-CSF antagonist inhibits the formation of such multinucleated cells.

In addition to the above co-culture systems, the ability of a candidate M-CSF antagonist in inhibiting osteoclastogenesis may be assayed in a stromal cell-free or osteoblast-free system. The M-CSF required for osteoclastogenesis may be provided by co-cultured metastatic cancer cells (e.g., MDA 231) or conditioned medium from these cancer cells (Mancino et al., J. Surg. Res. 0: 18-24, 2001) or by addition of purified M-CSF.

Efficacy of a given M-CSF antagonist in preventing or treating bone loss associated with cancer metastasis may also be tested in any of the animal bone metastasis model systems familiar to those skilled in the art. Such model systems include those involving direct injection of tumor cells into the medullary cavity of bones (Ingall, Proc. Soc. Exp. Biol. Med., 117: 819-22, 1964; Falasko, Clin. Orthop. 169: 20-7, 1982), into the rat abdominal aorta (Powles et al., Br. J. Cancer 28: 316-21, 1973), into the mouse lateral tail vein or into the mouse left ventricle (Auguello et al., Cancer Res. 48: 6876-81, 1988). In the absence of an effective M-CSF antagonist, osteolytic bone metastases formed from injected tumor cells may be determined by radiographs (areas of osteolytic bone lesions) or histochemistry (bone and soft tissues). Sasaki et al., Cancer Res. 55: 3551-7, 1995; Yoneda et al., J. Clin. Invest. 99: 2509-17, 1997. Clohisy and Ramnaraine, Orthop Res. 16: 660-6, 1998. Yin et al., J. Clin. Invest. 103: 197-206, 1999. In the presence of an effective M-CSF antigonist, osteolytic bone metastases may be prevented, or inhibited to result in fewer and/or smaller metastases.

The M-CSF antagonists of the present invention may also be useful in preventing or treating cancer metastasis. The effectiveness of a candidate M-CSF antagonist in preventing or treating cancer metastasis may be screened using a human amnionic basement membrane invasion model as described in Filderman et al., Cancer Res 52: 36616, 1992. In addition, any of the animal model systems for metastasis of various types of cancers may also be used. Such model systems include, but are not limited to, those described in Wenger et al., Clin. Exp. Metastasis 19: 169-73, 2002; Yi et al., Cancer Res. 62: 917-23, 2002; Tsutsumi et al., Cancer Lett 169: 77-85, 2001; Tsingotjidou et al., Anticancer Res. 21: 971-8, 2001; Wakabayashi et al., Oncology 59: 75-80, 2000; Culp and Kogerman, Front Biosci. 3:D672-83, 1998; Runge et al., Invest Radiol. 32: 212-7; Shioda et al., J. Surg. Oncol. 64: 122-6, 1997; Ma et al., Invest Ophthalmol Vis Sci. 37: 2293-301, 1996; Kuruppu et al., J Gastroenterol Hepatol. 11: 26-32, 1996. In the presence of an effective M-CSF antigonist, cancer metastases may be prevented, or inhibited to result in fewer and/or smaller metastases.

As provided herein, the compositions for and methods of treating cancer metastasis and/or bone loss associated with cancer metastasis may utilize one or more antibody used singularly or in combination with other therapeutics to achieve the desired effects. Antibodies according to the present invention may be isolated from an animal producing the antibody as a result of either direct contact with an environmental antigen or immunization with the antigen. Alternatively, antibodies may be produced by recombinant DNA methodology using one of the antibody expression systems well known in the art. See, e.g., Harlow and Lane, Antibodies: A Laboratory Manual, Cold Spring Harbor Laboratory (1988). Such antibodies may include recombinant IgGs, chimeric fusion proteins having immunoglobulin derived sequences or “humanized” antibodies that may all be used for the treatment of cancer metastasis and/or bone loss associated with cancer metastasis according to the present invention. In addition to intact, full-length molecules, the term antibody also refers to fragments thereof (such as, e.g., scFv, Fv, Fd, Fab, Fab′ and F(ab)′₂ fragments) or multimers or aggregates of intact molecules and/or fragments that bind to M-CSF. These antibody fragments bind antigen and may be derivatized to exhibit structural features that facilitate clearance and uptake, e.g., by incorporation of galactose residues.

In one embodiment of the present invention, M-CSF antagonists are monoclonal antibodies prepared essentially as described in Halenbeck et al. U.S. Pat. No. 5,491,065 (1997), incorporated herein by reference. Exemplary M-CSF antagonists include the monoclonal antibodies disclosed in this patent that bind to an apparent conformational epitope associated with recombinant or native dimeric M-CSF with concomitant neutralization of biological activity. These antibodies are substantially unreactive with biologically inactive forms of M-CSF including monomeric and chemically derivatized dimeric M-CSF.

In other embodiments of the present invention, humanized anti-M-CSF monoclonal antibodies are provided. The phrase “humanized antibody” refers to an antibody derived from a non-human antibody, typically a mouse monoclonal antibody. Alternatively, a humanized antibody may be derived from a chimeric antibody that retains or substantially retains the antigen binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are less immunogenic in humans than the parental mouse monoclonal antibodies, they can be used for the treatment of humans with far less risk of anaphylaxis. Thus, these antibodies may be preferred in therapeutic applications that involve in vivo administration to a human.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci., U.S.A., 81:6851-6855 (1984); Morrison and Oi, Adv. Immunol., 44:65 92 (1988); Verhoeyer et al., Science 239:1534-1536 (1988); Padlan, Molec. Immun. 28:489-498 (1991); Padlan, Molec. Immunol. 31(3):169-217 (1994); and Kettleborough, C. A. et al., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g., via Ashwell receptors. See, e.g., U.S. Pat. Nos. 5,530,101 and 5,585,089 which patents are incorporated herein by reference.

Humanized antibodies to M-CSF can also be produced using transgenic animals that are engineered to contain human immunoglobulin loci. For example, WO 98/24893 discloses transgenic animals having a human Ig locus wherein the animals do not produce functional endogenous immunoglobulins due to the inactivation of endogenous heavy and light chain loci. WO 91/741 also discloses transgenic non-primate mammalian hosts capable of mounting an immune response to an immunogen, wherein the antibodies have primate constant and/or variable regions, and wherein the endogenous immunoglobulin encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy chains, and express an exogenous immunoglobulin locus comprising one or more xenogeneic constant regions.

Using a transgenic animal described above, an immune response can be produced to a selected antigenic molecule, and antibody producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNFa, human CD4, L-selectin, gp39, and tetanus toxin. The monoclonal antibodies can be tested for the ability to inhibit or neutralize the biological activity or physiological effect of the corresponding protein. WO 96/3373 discloses that monoclonal antibodies against IL-8, derived from immune cells of transgenic mice immunized with IL-8, blocked IL-8 induced functions of neutrophils. Human monoclonal antibodies with specificity for the antigen used to immunize transgenic animals are also disclosed in WO 96/34096. The M-CSF antagonists of the present invention are said to be immunospecific or specifically binding if they bind to M-CSF with a Kₐ of greater than or equal to about 10⁴M⁻¹, preferably of greater than or equal to about 10⁵M⁻¹, more preferably of greater than or equal to about 10⁶M⁻¹ and still more preferably of greater than or equal to about 10⁷M⁻¹. Such affinities may be readily determined using conventional techniques, such as by equilibrium dialysis; by using the BIAcore 2000 instrument, using general procedures outlined by the manufacturer; by radioimmunoassay using ¹²⁵I-labeled M-CSF; or by another method known to the skilled artisan. The affinity data may be analyzed, for example, by the method of Scatchard et al., Ann N.Y. Acad. Sci., 51:660 (1949). Thus, it will be apparent that preferred M-CSF antagonists will exhibit a high degree of specificity for M-CSF and will bind with substantially lower affinity to other molecules.

Identification of additional M-CSF antagonists may be achieved by using any of a number of known methods for identifying and obtaining proteins that specifically interact with other proteins or polypeptides, for example, a yeast two-hybrid screening system such as that described in U.S. Pat. No. 5,283,173 or the equivalent may be utilized. In one embodiment of the present invention, a cDNA encoding M-CSF, or a fragment thereof, may be cloned into a two hybrid bait vector and used to screen a complementary target library for a protein having M-CSF binding activity.

As used herein, the term “protein” includes proteins, oligopeptides, polypeptides, peptides and the like. Additionally, the term protein may also refer to fragments, multimers or aggregates of intact molecules and/or fragments. Proteins may be naturally occurring or may be produced via recombinant DNA means or by chemical and/or enzymatic synthesis. See, e.g., Sambrook et al., Molecular Cloning: A Laboratory Manual, Cold Spring Harbor Laboratories (3rd ed. 2001).

In addition to antibodies and other proteins, this invention also contemplates alternative M-CSF antagonists including, but not limited to, small molecules that are also effective in treating cancer metastasis and/or bone loss associated with cancer metastasis. Such small molecules may be identified by assaying their capacity to bind to M-CSF and/or to inhibit the interaction between M-CSF and M-CSFR.

Methods for measuring the binding of M-CSF with small molecules are readily available in the art and include, for example, competition assays whereby the small molecule interferes with the interaction between M-CSF and its receptor (M-CSFR) or an anti M-CSF antibody. Alternatively, direct binding assays may be utilized to measure the interaction of a small molecule with M-CSF. By way of example, an ELISA assay may be employed whereby M-CSF is adsorbed onto an insoluble matrix such as a tissue culture plate or bead. A labeled M-CSFR or anti-M-CSF antibody is blocked from binding to M-CSF by inclusion of the small molecule of interest. Alternatively, the binding of a small molecule to M-CSF may be determined by a fluorescence activated cell sorting (FACS) assay. By this method, cells expressing M-CSF are incubated with a fluorescent tagged anti-M-CSF antibody or an anti-M-CSF antibody in the presence of a fluorescent tagged secondary antibody. Binding of a small molecule to M-CSF may be assessed by a dose dependent decrease in fluorescence bound to the M-CSF expressing cells. Similarly, direct binding of a small molecule may be assessed by labeling, e.g. radiolabeling or fluorescent tagging, the small molecule, incubating with immobilized M-CSF or M-CSF expressing cells and assaying for the radioactivity or fluorescence of the bound small molecule.

M-CSF antagonists of the present invention include, where applicable, functional equivalents. For example, molecules may differ in length, structure, components, etc. but may still retain one or more of the defined functions. More particularly, functional equivalents of the antibodies, antibody fragments or peptides of the present invention may include mimetic compounds, i.e., constructs designed to mimic the proper configuration and/or orientation for antigen binding.

Preferred M-CSF antagonists may optionally be modified by addition of side groups, etc., e.g., by amino terminal acylation, carboxy terminal amidation or by coupling of additional groups to amino acid side chains. Antagonists may also comprise one or more conservative amino acid substitutions. By “conservative amino acid substitutions” is meant those changes in amino acid sequence that preserve the general charge, hydrophobicity/hydrophilicity and/or steric bulk of the amino acid substituted. For example, substitutions between the following groups are conservative: Gly/Ala, Val/Ile/Leu, Asp/Glu, Lys/Arg, Asn/Gln, Ser/Cys/Thr, and Phe/Trp/Tyr. Such modifications will not substantially diminish the efficacy of the M-CSF antagonists and may impart such desired properties as, for example, increased in vivo half life or decreased toxicity.

Having identified more than one M-CSF antagonist that is effective in an animal model, it may be further advantageous to mix two or more such M-CSF antagonists together to provide still improved efficacy against cancer metastasis and/or bone loss associated with cancer metastasis. Compositions comprising one or more M-CSF antagonist may be administered to persons or mammals suffering from, or predisposed to suffer from, cancer metastasis and/or bone loss associated with cancer metastasis.

By the present methods, compositions comprising M-CSF antagonists may be administered parenterally, topically, orally or locally for therapeutic treatment. Preferably, the compositions are administered orally or parenterally, i.e., intravenously, intraperitoneally, intradermally or intramuscularly. Thus, this invention provides methods which employ compositions for administration which comprise one or more M-CSF antagonists in a pharmaceutically acceptable carrier, preferably an aqueous carrier. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

M-CSF antagonists useful as therapeutics for cancer metastasis or bone loss associated with cancer metastasis will often be prepared substantially free of other naturally occurring immunoglobulins or other biological molecules. Preferred M-CSF antagonists will also exhibit minimal toxicity when administered to a mammal afflicted with, or predisposed to suffer from, cancer metastasis and/or bone loss associated with cancer metastasis.

The compositions of the invention may be sterilized by conventional, well known sterilization techniques. The resulting solutions may be packaged for use or filtered under aseptic conditions and lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride and stabilizers (e.g., 1-20% maltose, etc.).

The M-CSF antagonists of the present invention may also be administered via liposomes. Liposomes, which include emulsions, foams, micelles, insoluble monolayers, phospholipid dispersions, lamellar layers and the like, can serve as vehicles to target the M-CSF antagonists to a particular tissue as well as to increase the half life of the composition. A variety of methods are available for preparing liposomes, as described in, e.g., U.S. Pat. Nos. 4,837,028 and 5,019,369, which patents are incorporated herein by reference.

The concentration of the M-CSF antagonist in these compositions can vary widely, i.e., from less than about 10%, usually at least about 25% to as much as 75% or 90% by weight and will be selected primarily by fluid volumes, viscosities, etc., in accordance with the particular mode of administration selected. Actual methods for preparing orally, topically and parenterally administrable compositions will be known or apparent to those skilled in the art and are described in detail in, for example, Remington's Pharmaceutical Science, 19^{th} ed., Mack Publishing Co., Easton, Pa. (1995), which is incorporated herein by reference.

Determination of an effective amount of a composition of the invention to treat cancer metastasis and/or bone loss associated with cancer metastasis in a patient can be accomplished through standard empirical methods which are well known in the art. For example, the in vivo neutralizing activity of sera from a subject treated with a given dosage of M-CSF antagonist may be evaluated using an assay that determines the ability of the sera to block M-CSF induced proliferation and survival of murine monocytes (CD11b+ cell, a subset of CD11 cells, which expresses high levels of receptor to M-CSF) in vitro as described in Cenci et al., J Clin. Invest. 1055: 1279-87, 2000.

Compositions of the invention are administered to a mammal already suffering from, or predisposed to, cancer metastasis and/or bone loss associated with cancer metastasis in an amount sufficient to prevent or at least partially arrest the development of cancer metastasis and/or bone loss associated with cancer metastasis. An amount adequate to accomplish this is defined as a “therapeutically effective dose.” Effective amounts of a M-CSF antagonist will vary and depend on the severity of the disease and the weight and general state of the patient being treated, but generally range from about 1.0 μg/kg to about 100 mg/kg body weight, with dosages of from about 10 μg/kg to about 10 mg/kg per application being more commonly used. Administration is daily, weekly or less frequently, as necessary depending on the response to the disease and the patient's tolerance of the therapy. Maintenance dosages over a prolonged period of time may be needed, and dosages may be adjusted as necessary.

Single or multiple administrations of the compositions can be carried out with the dose levels and pattern being selected by the treating physician. In any event, the formulations should provide a quantity of M-CSF antagonist sufficient to effectively prevent or minimize the severity of cancer metastasis and/or bone loss associated with cancer metastasis. The compositions of the present invention may be administered alone or as an adjunct therapy in conjunction with other therapeutics known in the art for the treatment of cancer metastasis and/or bone loss associated with cancer metastasis.

The following experimental examples are offered by way of illustration not limitation.

### EXAMPLES

### Example 1

This example shows that highly metastatic breast cancer cell lines express high levels of M-CSF. Using microarrays, the M-CSF gene expression by the highly metastatic cell line, MDA-231, was compared with that of the cell lines MCF7 and ZR751. There was a 6.9 fold increase when the M-CSF expression level in MDA-231 was compared with that in MCF7, and a 5.2 fold increase when the M-CSF expression level in MDA-231 was compared with that in ZR751.

### Example 2

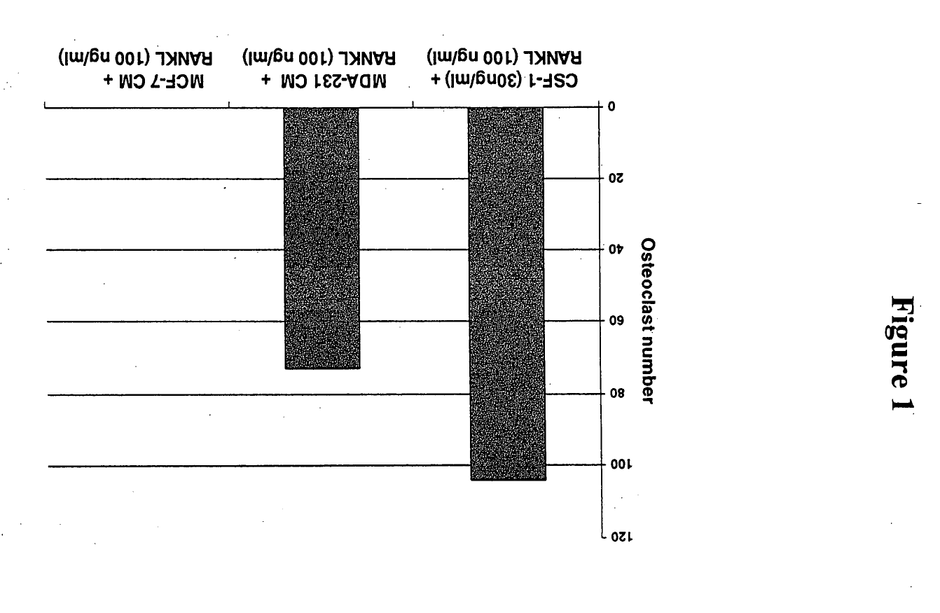

This example shows that purified M-CSF can be replaced by conditioned media (CM) from the metastatic cell line MDA-231 but not from the cell line MCF7 in in vitro assays of osteoclast formation (FIG. 1).

Production of conditioned media (CM): MDA231 or MCF7 cells were plated at a density of 1×10⁶ cells/10 cm dish in 8 mls of 50% DMEM/50% HAMs F12 containing 1×ITS, available from BD Biosciences located in Lexington, Ky., USA, a culture supplement containing insulin, human transferrin, and selenous acid. After 48 hours of incubation at 37° C. in 5% CO₂, the media were collected and centrifuged for 10 minutes at 1500 RPM to remove any suspended cells. The supernatant was collected, filtered through a 0.2 nM filter and used as CM.

Osteoclast assay: Bone marrow CD34+ cells were plated at a density of 15,000 cells/96 well in 100 μl of Alpha MEM containing 10% FCS, 1×Pen/Strep and 1×fungizone. The next day, 50 μl of media was removed from each well and replaced with 25 μl of Alpha MEM media and 75 μl of CM or 50% DMEM/50% HAMs F12 containing 1×ITS. RANKL was added to each well at a final concentration of 100 ng/ml and 30 ng/ml M-CSF was added to the appropriate wells. The cells were incubated at 37° C. in 5% CO₂ for 11 days. During that time fresh RANKL was added again after 6 days. After 11 days the cells were fixed and stained for tartrate resistant acid phosphatase using the Leukocyte acid phosphatase kit from Sigma.

### Example 3

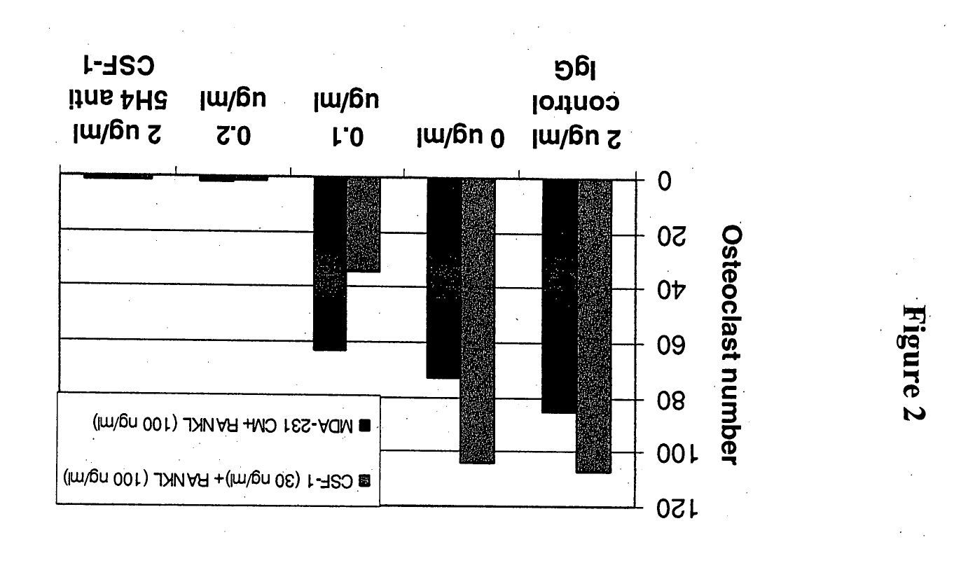

This example shows that osteoclast induction by MDA-231 CM is neutralized by antibodies to M-CSF (FIG. 2).

Bone marrow CD34+ cells were plated as described in Example 2. The next day 50 μl of media was removed from each well. 25 μl of 6× antibody or Alpha MEM media was added to each well followed by 75 μl of CM or 50% DMEM/50% HAMs F12 containing 1×ITS or alpha. MEM media. 100 ng/ml RANKL was added to all wells, and 30 ng/ml M-CSF was added to half of the wells. The cells were incubated at 37° C. in 5% CO₂ for 11 days. During that time fresh RANKL was added again after 6 days. After 11 days, the cells were fixed and stained for tartrate resistant acid phosphatase using the Leukocyte acid phosphatase kit from Sigma.

### Example 4

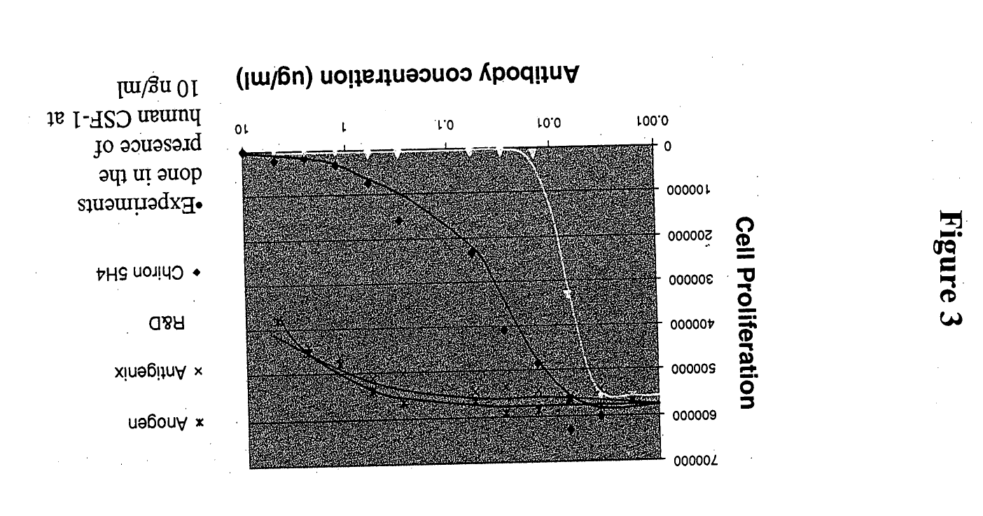

This example shows neutralizing activity of a monoclonal antibody 5H4 (American Type Culture Collection Accession No. HB10027) and other antibodies against human M-CSF (FIG. 3).

To measure the neutralizing ability of the antibodies against the activity of human M-CSF on murine M-NFS-60 cells (American Type Culture Collection Accession No. CRL-1838, available from ATCC in Rockville, Md., USA, derived from a myelogenous leukemia induced with the Cas-Br-MuLV wild mouse ecotropic retrovirous, responsive to both interleukin 3 and M-CSF and which contain a truncated c-myb proto-oncogene caused by the integration of a retrovirus), recombinant human CSF-1 (at 10 ng/ml final concentration) was incubated with various concentrations of antibodies for 1 hour at 37° C. CO₂ in an incubator. Following the incubation, the mixture was added to the M-NFS-60 culture in 96-well microtiter plates. The total assay volume per well was 100 μl, with 10 ng/ml rhM-CSF, indicated antibody concentration, and cell density at 5,000 cells/well. After 72 hours culture in a CO₂ incubator at 37° C., cell proliferation was assayed by CellTiter Glo Kit (Promega). All antibodies were raised against human M-CSF. Anogen refers to Anogen product catalog #MO-C40048.A clone 116; Antigenix refers to Antigenix America product catalog #MC600520 clone M16; and R&D refers to R&D Systems product catalog #Mab216.

All of the above U.S. patents, U.S. patent application publications, U.S. patent applications, foreign patents, foreign patent applications and non patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A method for treating a mammal afflicted with a metastatic cancer to bone comprising administering to said mammal a therapeutically effective amount of M-CSF antagonist thereby reducing the severity of bone loss associated with the metastatic cancer.
